SEQUENCE LISTING (17 sequences)

SEQ ID NO: 1 - RNA, 22 nt, synthetic construct
  source 1..22: mol_type = other RNA
tagcttatcagactgatgttga

SEQ ID NO: 2 - RNA, 21 nt, synthetic construct
  source 1..21: mol_type = other RNA
caacaccagtcgatgggctgt

SEQ ID NO: 3 - RNA, 22 nt, synthetic construct
  source 1..22: mol_type = other RNA
tagcaccatctgaaatcggtta

SEQ ID NO: 4 - RNA, 23 nt, synthetic construct
  source 1..23: mol_type = other RNA
tagcaccatttgaaatcagtgtt

SEQ ID NO: 5 - RNA, 22 nt, synthetic construct
  source 1..22: mol_type = other RNA
tagcaccatttgaaatcggtta

SEQ ID NO: 6 - RNA, 22 nt, synthetic construct
  source 1..22: mol_type = other RNA
tgagaactgaattccatgggtt

SEQ ID NO: 7 - RNA, 22 nt, synthetic construct
  source 1..22: mol_type = other RNA
cctctgaaattcagttcttcag

SEQ ID NO: 8 - protein, 7 aa, synthetic construct
QHREDGS

SEQ ID NO: 9 - protein, 20 aa, synthetic construct
HISDPHEARGTRPDPHELYS

SEQ ID NO: 10 - protein, 13 aa, synthetic construct
IWSLDTQYGGRGD

SEQ ID NO: 11 - protein, 13 aa, synthetic construct
IWSLDTQYGGRGD

SEQ ID NO: 12 - protein, 13 aa, synthetic construct
IWSLDTQYGGRGD

SEQ ID NO: 13 - protein, 9 aa, synthetic construct
MPFRWFKPV

SEQ ID NO: 14 - protein, 9 aa, synthetic construct
MPFRWFKPV

SEQ ID NO: 15 - protein, 15 aa, synthetic construct
RADARADARADARAD

SEQ ID NO: 16 - protein, 12 aa, synthetic construct
KLDLKLDLKLDL

SEQ ID NO: 17 - protein, 11 aa, synthetic construct
QQKFQFQFEQQ

(19) United States
(12) Patent Application Publication
(10) Pub. No.: US 2026/0240911 A1
(43) Pub. Date: Aug. 20, 2026

(54) Compositions and Methods for Peptide-Exosome Hybrid Wound Healing, Scar Prevention, and Post-Procedure Skin Recovery Using Placental Extracts and Biocompatible Scaffolds

(71) Applicants: Nicholas Jerome Andrews, Flanders, NJ (US); Roy Charles Korth, Grove City, OH (US)

(72) Inventors: Nicholas Jerome Andrews, Flanders, NJ (US); Roy Charles Korth, Grove City, OH (US)

(21) Appl. No.: 19/531,986

(22) Filed: Feb. 6, 2026

Related U.S. Application Data
(60) Provisional application No. 63/755,521, filed on Feb. 7, 2025.

(51) Int. Cl. A61K 35/28; A61K 9/06; A61K 31/045; A61K 31/164; A61K 31/195; A61K 31/355; A61K 31/4015; A61K 31/455; A61K 31/722; A61K 31/728; A61K 33/30; A61K 36/23; A61K 38/05; A61K 38/06; A61K 38/07; A61K 38/08; A61K 38/10

(52) U.S. Cl. CPC A61K 35/28; A61K 9/06; A61K 31/045; A61K 31/164; A61K 31/195; A61K 31/355; A61K 31/4015; A61K 31/455; A61K 31/722; A61K 31/728; A61K 33/30; A61K 36/23; A61K 38/05; A61K 38/06; A61K 38/07; A61K 38/08; A61K 38/10

(57) ABSTRACT

The present disclosure provides compositions, kits, and methods for enhanced wound healing, scar prevention, and post-procedural skin recovery. The compositions co-localize exosomes and a multi-peptide complex within a placental ECM-derived scaffold to provide coordinated modulation of inflammatory, proliferative, and remodeling pathways and controlled topical delivery.

### REFERENCE TO ELECTRONIC SEQUENCE LISTING

The application contains a Sequence Listing which has been submitted electronically in .XML format and is hereby incorporated by reference in its entirety. Said .XML copy, created on Feb. 6, 2026, is named “PushPatch-003US.xml” and is 15,683 bytes in size. The sequence listing contained in this .XML file is part of the specification and is hereby incorporated by reference herein in its entirety.

### CROSS REFERENCE TO RELATED APPLICATIONS

The present application claims the benefit of the filing date of U.S. Provisional Patent Application No. 63/755,521, filed on Feb. 7, 2025, the disclosure of which is hereby incorporated by reference herein in its entirety.

### FIELD OF THE DISCLOSURE

The present disclosure relates to regenerative biomaterials and dermatologic therapeutics. More particularly, it relates to topical compositions and methods for wound healing, fibrotic scar prevention or reduction, and post-procedure dermal recovery, including after aesthetic or dermatologic procedures. In some embodiments, the disclosed systems employ a hybrid platform integrating extracellular vesicle signaling (e.g., exosomes), defined peptide modulators, placental-derived extracellular matrix (ECM) components, and tunable biocompatible scaffolds configured to regulate cellular and molecular wound responses.

### BACKGROUND OF THE DISCLOSURE

Cutaneous wound healing proceeds through overlapping inflammatory, proliferative, and remodeling phases. Pathologic scarring (e.g., hypertrophic scars and keloids) can arise from persistent inflammation, dysregulated fibroblast activity, excessive extracellular matrix deposition, and aberrant mechanotransduction.

Despite extensive advances in wound care and scar management, current technologies frequently fall short in providing coordinated control over the inflammatory, proliferative, and remodeling phases of healing. Traditional topical agents, procedural interventions, and biologic therapeutics demonstrate significant limitations in bioavailability, targeted activity, and durability of effect.

Conventional topical approaches (e.g., silicone gels, antioxidants, isolated growth factors) may provide barrier or symptomatic benefits but often fail to coordinate modulation of inflammation, angiogenesis, fibroblast phenotype, and extracellular matrix remodeling. Procedural approaches (e.g., ablative, or fractional lasers, intralesional corticosteroids, surgical excision) may improve appearance in some cases but can be associated with recurrence, pigmentary changes, dermal atrophy, or incomplete normalization of profibrotic signaling.

Extracellular vesicles, including mesenchymal stromal/stem cell (MSC)-derived exosomes, have shown immunomodulatory and regenerative potential; however, topical, or local delivery may suffer from limited retention, diffusion, and stability when not provided within a structural matrix. Similarly, placental-derived extracts may contain ECM proteins and growth factors but can be unstable or insufficiently localized without a compatible scaffold. Accordingly, there remains a need for compositions and delivery systems that provide coordinated, localized, and temporally sustained regulation of inflammatory signaling, fibroblast activation state, angiogenesis, and/or matrix remodeling to support rapid healing with reduced scarring.

### BRIEF SUMMARY OF THE DISCLOSURE

The present disclosure provides compositions and methods that invention addresses these shortcomings by co-delivering mechanistically complementary agents in a scaffolded hydrogel platform, engineered for synergistic control over key cellular and extracellular wound healing parameters. It is believed that the disclosed compositions integrate (1) exosome-mediated signaling (e.g., immunomodulatory and regenerative miRNA and protein cargo), (2) defined peptide modulators selected to influence extracellular matrix turnover, inflammation, mechanotransduction, angiogenesis, and/or pigmentation, and (3) a placental ECM-derived scaffold that provides hydration, mechanical compliance, and spatial retention of the actives.

Without wishing to be bound by any particular theory, it is believed that the coordinated delivery of these components can provide multi-axis modulation across the wound healing cascade, including down-regulation of profibrotic pathways (e.g., TGF-β/Smad), suppression of inflammatory pathways (e.g., NF-κB), regulation of matrix metalloproteinase (MMP) and tissue inhibitor of metalloproteinase (TIMP) balance, attenuation of mechanotransductive signaling (e.g., YAP/TAZ-mediated myofibroblast activation), and improvement of post-inflammatory pigment outcomes.

One aspect of the present disclosure is a composition comprising: (i) a population of extracellular vesicles comprising exosomes; (ii) a peptide complex comprising two or more peptides that modulate one or more of extracellular matrix remodeling, inflammation, mechanotransduction, angiogenesis, or pigmentation; and (iii) a scaffold comprising placental-derived extracellular matrix components, wherein the exosomes and peptide complex are co-localized within the scaffold to increase dermal retention and coordinate activity.

In some embodiments, the scaffold is a placental MSC-derived exosomes. In some embodiments, the exosomes include placental MSC-derived exosomes enriched with miR-21, miR-29, and/or miR-146a. In some embodiments, the scaffold offers controlled release, such as for a time period ranging from between about 24 hours to about 72 hours.

In some embodiments, the peptide complex includes (i) one or more copper-binding/ECM-modulating peptides; (ii) one or more fibrosis-modulating/anti-TGF-β peptides; (iii) one or more mechanotransduction/contraction inhibitors; and/or (iv) one or more angiogenesis-modulating Peptides. In some embodiments, the peptide composition further includes one or more pigmentation-regulating and depigmenting peptides. In some embodiments, the peptide composition further includes one or more wound healing peptides.

In some embodiments, the peptide complex comprising two or more peptides selected from GHK-Cu, Tetrapeptide-7, Palmitoyl Tripeptide-1, Acetyl Hexapeptide-8, Oligopeptide-20, Myristoyl Hexapeptide-16, and Oligopeptide-34. In some embodiments, the peptide complex comprising three or more peptides selected from GHK-Cu, Tetrapeptide-7, Palmitoyl Tripeptide-1, Acetyl Hexapeptide-8, Oligopeptide-20, Myristoyl Hexapeptide-16, and Oligopeptide-34. In some embodiments, the peptide complex comprising four or more peptides selected from GHK-Cu, Tetrapeptide-7, Palmitoyl Tripeptide-1, Acetyl Hexapeptide-8, Oligopeptide-20, Myristoyl Hexapeptide-16, and Oligopeptide-34. In some embodiments, the peptide complex comprising five or more peptides selected from GHK-Cu, Tetrapeptide-7, Palmitoyl Tripeptide-1, Acetyl Hexapeptide-8, Oligopeptide-20, Myristoyl Hexapeptide-16, and Oligopeptide-34.

In some embodiments, at least one peptide of the two or more peptides of the peptide complex are modified by one of: (i) PEGylation with polyethylene glycol chains of variable length; (ii) lipidation with a lipid moiety selected from palmitoyl, myristoyl, stearoyl, lauroyl, or oleoyl; (iii) cyclization of the peptide, including head-to-tail cyclization or side-chain stapling; (iv) substitution of one or more D-amino acids at non-critical positions; and (v) selective N-methylation of peptide bonds; wherein the modification enhances at least one of peptide stability, bioavailability, or therapeutic activity.

In some embodiments, at least one peptide of the two or more peptides of the peptide complex are conjugated to a biodegradable polymer selected from poly(lactic-co-glycolic acid) (PLGA) or hyaluronic acid via a cleavable linker configured to enable sustained release. In some embodiments, the cleavable linker is an enzyme-responsive sequence cleavable by a wound-associated enzyme selected from matrix metalloproteinases (MMPs). In some embodiments, at least one peptide of the two or more peptides of the peptide complex includes a hydrocarbon staple configured to stabilize an α-helical conformation. In some embodiments, at least one peptide of the two or more peptides of the peptide complex makes a bifunctional sequence including both an anti-inflammatory domain and an extracellular matrix-remodeling domain. In some embodiments, at least one peptide of the two or more peptides of the peptide complex is a GHK-based peptide conjugated to form a self-assembling nanostructure providing a depot effect. In some embodiments, at least one peptide of the two or more peptides of the peptide complex is a variant of Acetyl Hexapeptide-8 including an alternative acyl group selected from benzoyl or isobutyryl. In some embodiments, at least one peptide of the two or more peptides of the peptide complex is a variant of Tetrapeptide-7 including a sequence extension or cyclization configured to enhance anti-fibrotic activity. In some embodiments, at least one peptide of the two or more peptides of the peptide complex includes a depigmenting sequence conjugated to a tranexamic acid analog via a linker optimized for controlled release. In some embodiments, at least one peptide of the two or more peptides of the peptide complex is a VEGF-mimetic oligopeptide configured as a dimerized or cyclized construct to mimic VEGF multimeric structure while reducing off-target angiogenesis.

A second aspect of the present disclosure is a composition for topical application to promote wound healing and scar reduction, the composition comprising: (a) a population of exosomes derived from mesenchymal stem cells (MSCs); (b) a peptide complex comprising two or more peptides selected from GHK-Cu, Tetrapeptide-7, Palmitoyl Tripeptide-1, Acetyl Hexapeptide-8, Oligopeptide-20, Myristoyl Hexapeptide-16, and Oligopeptide-34; and (c) a biocompatible scaffold comprising placental-derived extracellular matrix (ECM) components selected from Type I, Type III, and Type IV collagens, fibronectin, and laminin; wherein the exosomes and the peptide complex are co-localized within the scaffold.

In some embodiments, the MSCs are enriched with one or more microRNAs selected from the group consisting of miR-21, miR-29, and miR-146a. In some embodiments, the exosomes are present at a concentration of about 1×10⁹ to 1×10¹¹ particles per milliliter. In some embodiments, the peptide complex comprises three or more of the peptides. In some embodiments, the peptide complex comprises four or more of the peptides. In some embodiments, the peptide complex includes: GHK-Cu at about 0.10% to 3.00% w/w; Tetrapeptide-7 at about 0.50% to 2.00% w/w; Palmitoyl Tripeptide-1 at about 1.00% to 3.00% w/w; Acetyl Hexapeptide-8 at about 0.50% to 2.00% w/w; and Oligopeptide-20 at about 0.25% to 1.00% w/w.

In some embodiments, the scaffold further comprises one or more placental growth factors. In some embodiments, the one or more placetal growth factors are selected from the group consisting of VEGF, PDGF, EGF, and HGF. In some embodiments, the one or more placental growth factors are each present at about 5 ng/mL to about 50 ng/mL in the scaffold or final composition. In some embodiments, the scaffold is in the form of a hydrogel, gel-cream, emulsion, or other dermatologically acceptable topical base. In some embodiments, the scaffold is heat-treated or decellularized placental ECM providing controlled release of exosomes and peptides.

In some embodiments, the composition further comprisesone or more additional agents selected from: one or more antioxidants, one or more humectants, one or more antimicrobials, one or more barrier lipids, one or more pigmentation regulators, and one or moresoothing botanicals. In some embodiments, the one or more antioxidants is carnosine. In some embodiments, the one or more humectants is hyaluronic acid. In some embodiments, the one or more antimicrobials is chitosan. In some embodiments, the one or more barrier lipids is a ceramide. In some embodiments, the one or more pigmentation regulators are tranexamic acid or oligopeptide-34. In some embodiments, the one or more soothing botanticals are Centella asiatica extract or bisabolol.

In some embodiments, at least one peptide of the two or more peptides of the peptide complex are conjugated to a biodegradable polymer selected from poly(lactic-co-glycolic acid) (PLGA) or hyaluronic acid via a cleavable linker configured to enable sustained release. In some embodiments, the cleavable linker is an enzyme-responsive sequence cleavable by a wound-associated enzyme selected from matrix metalloproteinases (MMPs). In some embodiments, at least one peptide of the two or more peptides of the peptide complex includes a hydrocarbon staple configured to stabilize an α-helical conformation. In some embodiments, at least one peptide of the two or more peptides of the peptide complex makes a bifunctional sequence including both an anti-inflammatory domain and an extracellular matrix-remodeling domain. In some embodiments, at least one peptide of the two or more peptides of the peptide complex is a GHK-based peptide conjugated to form a self-assembling nanostructure providing a depot effect. In some embodiments, at least one peptide of the two or more peptides of the peptide complex is a variant of Acetyl Hexapeptide-8 including an alternative acyl group selected from benzoyl or isobutyryl. In some embodiments, at least one peptide of the two or more peptides of the peptide complex is a variant of Tetrapeptide-7 including a sequence extension or cyclization configured to enhance anti-fibrotic activity. In some embodiments, at least one peptide of the two or more peptides of the peptide complex includes a depigmenting sequence conjugated to a tranexamic acid analog via a linker improved for controlled release. In some embodiments, at least one peptide of the two or more peptides of the peptide complex is a VEGF-mimetic oligopeptide configured as a dimerized or cyclized construct to mimic VEGF multimeric structure while reducing off-target angiogenesis.

A third aspect of the present disclosure is a kit for post-procedure dermatologic recovery, comprising: (a) a first component comprising placental MSC-derived exosomes; (b) a second component comprising a peptide complex and a scaffold matrix; and (c) instructions for combining the first and second components immediately prior to application.

A fourth aspect of the present disclosure is a method of treating a cutaneous wound or reducing the risk of fibrotic scarring in a subject, comprising: topically applying to the wound site a composition comprising: (a) a population of exosomes derived from mesenchymal stem cells (MSCs); (b) a peptide complex comprising two or more peptides selected from GHK-Cu, Tetrapeptide-7, Palmitoyl Tripeptide-1, Acetyl Hexapeptide-8, Oligopeptide-20, Myristoyl Hexapeptide-16, and Oligopeptide-34; and (c) a biocompatible scaffold comprising placental-derived extracellular matrix (ECM) components selected from Type I, Type III, and Type IV collagens, fibronectin, and laminin; wherein the exosomes and the peptide complex are co-localized within the scaffold, wherein the composition modulates inflammatory signaling, promotes extracellular matrix remodeling, and reduces TGF-beta and YAP/TAZ-mediated mechanotransduction pathways.

A fifth aspect of the present disclosure is a composition comprising: (a) a population of extracellular vesicles comprising exosomes; (b) a peptide complex comprising two or more peptides that modulate at least one of inflammation, extracellular matrix remodeling, mechanotransduction, angiogenesis, or pigmentation; and (c) a biocompatible scaffold comprising placental-derived extracellular matrix (ECM) components; wherein the exosomes and the peptide complex are co-localized within the scaffold.

In some embodiments, the exosomes are derived from mesenchymal stromal/stem cells (MSCs). In some embodiments, the MSCs comprise placental-derived MSCs, umbilical cord-derived MSCs, Wharton's jelly MSCs, adipose-derived MSCs, or bone marrow-derived MSCs. In some embodiments, the exosomes are enriched with one or more microRNAs selected from mi-R21, miR-29, and miR-146a. In some embodiments, the exosomes are present at about 1×10\hat{8} to about 5×10\hat{ }11 particles per mL. In some embodiments, the exosomes are present at about 1×10\hat{ }9 to about 1×10\hat{ }11 particles per mL.

In some embodiments, the peptide complex comprises three or more peptides. In some embodiments, the peptide complex includes four or more peptides. In some embodiments, the peptide complex includes five or more peptides. In some embodiments, the peptide complex includes at least one copper-binding peptide selected from GHK-Cu and Palmitoyl-composition GHK. In some embodiments, the peptide complex includes at least one anti-fibrotic peptide selected from the group consisting of Tetrapeptide-7 and a decorin-mimetic peptide. In some embodiments, the peptide complex includes at least one angiogenesis-modulating peptide selected from the group consisting of Oligopeptide-20 and QK peptide. In some embodiments, the peptide complex further includes a pigmentation-regulating peptide selected from the group consisting of Oligopeptide-34 and Hexapeptide-9. In some embodiments, the peptide complex comprises two or more peptides selected from the group consisting of GHK-Cu, Tetrapeptide-7, Palmitoyl Tripeptide-1, Palmitoyl Pentapeptide-4, Acetyl Hexapeptide-8, Oligopeptide-20, Myristoyl Hexapeptide-16, Oligopeptide-34, QK peptide, and thymosin-β4.

In some embodiments, at least one peptide of the two or more peptides of the peptide complex are conjugated to a biodegradable polymer selected from poly(lactic-co-glycolic acid) (PLGA) or hyaluronic acid via a cleavable linker configured to enable sustained release. In some embodiments, the cleavable linker is an enzyme-responsive sequence cleavable by a wound-associated enzyme selected from matrix metalloproteinases (MMPs). In some embodiments, at least one peptide of the two or more peptides of the peptide complex includes a hydrocarbon staple configured to stabilize an α-helical conformation. In some embodiments, at least one peptide of the two or more peptides of the peptide complex makes a bifunctional sequence including both an anti-inflammatory domain and an extracellular matrix-remodeling domain. In some embodiments, at least one peptide of the two or more peptides of the peptide complex is a GHK-based peptide conjugated to form a self-assembling nanostructure providing a depot effect. In some embodiments, at least one peptide of the two or more peptides of the peptide complex is a variant of Acetyl Hexapeptide-8 including an alternative acyl group selected from benzoyl or isobutyryl. In some embodiments, at least one peptide of the two or more peptides of the peptide complex is a variant of Tetrapeptide-7 including a sequence extension or cyclization configured to enhance anti-fibrotic activity. In some embodiments, at least one peptide of the two or more peptides of the peptide complex includes a depigmenting sequence conjugated to a tranexamic acid analog via a linker optimized for controlled release. In some embodiments, at least one peptide of the two or more peptides of the peptide complex is a VEGF-mimetic oligopeptide configured as a dimerized or cyclized construct to mimic VEGF multimeric structure while reducing off-target angiogenesis.

In some embodiments, the peptide complex comprises: GHK-Cu at about 0.01% to about 3.00% w/w; Tetrapeptide-7 at about 0.01% to about 2.00% w/w; Palmitoyl Tripeptide-1 at about 0.05% to about 5.00% w/w; Acetyl Hexapeptide-8 at about 0.01% to about 2.00% w/w; and Oligopeptide-20 at about 0.01% to about 2.00% w/w.

In some embodiments, the two or more peptides in the peptide complex are lipidated, PEGylated, cyclized, D-amino-acid substituted, N-methylated, or encapsulated in liposomes.

In some embodiments, the placental-derived ECM components comprise one or more collagens selected from Type I collagen, Type III collagen, and Type IV collagen, and further comprise fibronectin and laminin. In some embodiments, the scaffold comprises a placental ECM-derived hydrogel. In some embodiments, the scaffold further comprises one or more placental growth factors selected from VEGF, PDGF, EGF, and HGF. In some embodiments, the hydrogel is crosslinked using genipin, EDC/NHS chemistry, transglutaminase, photo-crosslinking, or ionic crosslinking. In some embodiments, the scaffold provides controlled release of the exosomes and peptides for at least 4 hours. In some embodiments, the scaffold provides controlled release for at least 24 hours. In some embodiments, the scaffold provides controlled release for at least 72 hours. In some embodiments, the composition is formulated as a hydrogel, gel-cream, emulsion, ointment, lotion, spray, foam, or serum.

In some embodiments, the composition is formulated as a patch, film, or dressing impregnated with the exosomes and peptide complex.

In some embodiments, the composition further comprises one or more additional agents selected from antioxidants, humectants, antimicrobials, barrier lipids, pigmentation regulators, anesthetics, and soothing botanicals. In some embodiments, the one or more additional agents comprise one or more of carnosine, hyaluronic acid, chitosan, ceramides, niacinamide, centella asiatica extract, bisabolol, vitamin E, zinc PCA, tranexamic acid, panthenol, and allantoin.

In some embodiments, the composition has a pH of about 4.5 to about 7.5.

A sixth aspect of the present disclosure is a method of treating a cutaneous wound in a subject, comprising topically applying to a wound site a composition comprising: (a) a population of extracellular vesicles comprising exosomes; (b) a peptide complex comprising two or more peptides that modulate at least one of inflammation, extracellular matrix remodeling, mechanotransduction, angiogenesis, or pigmentation; and (c) a biocompatible scaffold comprising placental-derived extracellular matrix (ECM) components; wherein the exosomes and the peptide complex are co-localized within the scaffold. In some embodiments, the wound comprises a surgical incision, abrasion, laceration, burn, or chronic ulcer. In some embodiments, the wound site comprises a post-procedure site selected from a laser resurfacing site, microneedling site, dermabrasion site, radiofrequency treatment site, ultrasound treatment site, and chemical peel site. In some embodiments, the composition is applied within 0 to 24 hours after the procedure and then applied once or twice daily for at least 3 days. In some embodiments, the application reduces one or more of TGF-β signaling, NF-κB signaling, YAP/TAZ signaling, α-SMA expression, or collagen gel contraction relative to an untreated control. In some embodiments, the application reduces incidence or severity of hypertrophic scarring or keloid formation. In some embodiments, the application reduces post-inflammatory hyperpigmentation or prolonged erythema following the procedure.

### DETAILED DESCRIPTION

It should also be understood that, unless clearly indicated to the contrary, in any methods claimed herein that include more than one step or act, the order of the steps or acts of the method is not necessarily limited to the order in which the steps or acts of the method are recited.

As used herein, the singular terms “a,” “an,” and “the” include plural referents unless context clearly indicates otherwise. Similarly, the word “or” is intended to include “and” unless the context clearly indicates otherwise. The term “includes” is defined inclusively, such that “includes A or B” means including A, B, or A and B.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of” or “exactly one of.” “Consisting essentially of,” when used in the claims, shall have its ordinary meaning as used in the field of patent law.

The terms “comprising,” “including,” “having,” and the like are used interchangeably and have the same meaning. Similarly, “comprises,” “includes,” “has,” and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a device having components a, b, and c” means that the device includes at least components a, b, and c. Similarly, the phrase: “a method involving steps a, b, and c” means that the method includes at least steps a, b, and c. Moreover, while the steps and processes may be outlined herein in a particular order, the skilled artisan will recognize that the ordering steps and processes may vary.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Reference throughout this specification to “one embodiment” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein, “exosome” refers to a subset of extracellular vesicles (EVs) typically having diameters of about 30-200 nm, including vesicles enriched for proteins and nucleic acids that mediate intercellular signaling. The term “extracellular vesicles” includes exosomes and other vesicles (e.g., microvesicles) unless otherwise specified.

As used herein, “scaffold” refers to a biocompatible matrix (e.g., hydrogel, gel-cream, emulsion, nanofiber matrix, film, patch, dressing, or ECM-derived gel) that provides structural support and/or retention of one or more active agents at a treatment site.

As used herein, “wound” includes acute and chronic cutaneous wounds, including surgical incisions, abrasions, lacerations, burns, laser- or energy-device induced micro-injury, microneedling-induced injury, chemical peel injury, and other iatrogenic or traumatic skin injuries.

As used herein, “scar prevention” includes reducing the incidence, severity, thickness, erythema, pigmentation change, contracture, pruritus, and/or recurrence risk of hypertrophic scars and keloids relative to an untreated control or standard of care.

As used herein, “co-localized” or “co-immobilized” refers to physical association of exosomes and peptides with a scaffold (e.g., by entrapment within a hydrogel network, adsorption to ECM proteins, ionic complexation, covalent tethering via releasable linkers, or encapsulation within secondary carriers embedded in the scaffold), such that the actives exhibit increased residence time at the treatment site compared to delivery without the scaffold.

### Overview

The present disclosure provides compositions and methods for enhanced wound healing, scar prevention, and post-procedural skin recovery. In some embodiments, the compositions synergistically integrate MSC-derived exosomes (optionally enriched with selected microRNAs), a defined bioactive peptide complex, and a biocompatible scaffold comprising one or more placental ECM components. Without wishing to be bound by any theory, it is believed that the disclosed compositions promote coordinated modulation of inflammation, extracellular matrix remodeling, and angiogenesis while suppressing pro-fibrotic and mechanotransductive signaling pathways.

In some embodiments, the exosomes and peptide complex (e.g., a combination of two or more peptides, three or more peptides, four or more peptides, five or more peptides, etc.) are co-immobilized or co-localized within a thermally stabilized or crosslinked hydrogel, gel-cream, or film to provide dermal retention and sustained release. In some embodiments, the peptide complex includes ECM modulating and anti-fibrotic agents such as GHK-Cu, Tetrapeptide-7, Acetyl Hexapeptide-8, and Oligopeptide-20. In some embodiments, placental ECM components (collagens I, III, IV, fibronectin, laminin) and embedded growth factors provide structural support.

In some embodiments, the compositions are configured to (i) reduce inflammatory cytokine signaling (e.g., IL-6, TNF-α), (ii) promote keratinocyte migration and re-epithelialization, (iii) bias macrophage polarization toward pro-resolving phenotypes, (iv) reduce myofibroblast differentiation and contractile force generation, and/or (v) normalize collagen architecture and pigmentation during remodeling.

Also disclosed herein are methods for treating wounds include topical application of the composition to promote regenerative healing with reduced scarring. Without wishing to be bound by any theory, it is believed that the multi-pathway, scaffold-enhanced platform advances the art by providing sustained, spatially localized delivery of bioactives tailored for clinical dermatologic regeneration.

### Compositions

In view of the foregoing, one aspect of the present disclosure is a composition comprising: (i) a population of exosomes; (ii) a peptide complex; and (iii) a scaffold comprising placental-derived extracellular matrix components; wherein the exosomes and peptide composition are co-localized within the scaffold.

### Exosomes

In some embodiments, the exosomes are derived from a regenerative cell source such as mesenchymal stromal/stem cells (MSCs), including placental-derived MSCs, umbilical cord-derived MSCs, Wharton's jelly MSCs, adipose-derived MSCs, or bone marrow-derived MSCs.

In some embodiments, the exosomes are isolated from conditioned media using differential centrifugation, ultrafiltration, tangential-flow filtration, size exclusion chromatography, precipitation, immunoaffinity capture, or combinations thereof.

In some embodiments, the exosomes have a mean diameter of about 30-200 nm as measured by nanoparticle tracking analysis (NTA) or dynamic light scattering (DLS). In some embodiments, the exosomes are characterized by one or more markers selected from CD9, CD63, CD81, TSG101, and Alix, and have low levels of non-vesicular contaminants (e.g., albumin) relative to total protein.

In some embodiments, the exosomes are mesenchymal stem cell-derived exosomes. In some embodiments, mesenchymal stem cell-derived exosomes are enriched with one or more microRNA species (such as those associated with wound healing modulation). In some embodiments, the microRNA species include, but are not limited to, miR-21, miR-29, and miR-146a (see, e.g., SEQ ID NOS. 1-7). In some embodiments, enrichment is achieved by culturing source cells under defined conditions (e.g., hypoxia, cytokine conditioning) or by loading selected nucleic acids into exosomes.

In some embodiments, the exosomes are provided at about 1×10\hat{ }8 to about 5×10\hat{ }11 particles per mL, such as at about 1×10\hat{ }9 to about 1×10\hat{ }11 particles per mL; or an equivalent unit-dose particle count (e.g., per 5 mL, 10 mL, 30 mL, or per patch/dressing). In some embodiments, the exosome preparation corresponds to a total protein concentration of about 1 μg/mL to about 500 μg/mL.

In some embodiments, the exosomes are provided as an aqueous suspension. In other embodiments, the exosomes are provided as a lyophilized powder, optionally with one or more cryoprotectants (e.g., trehalose, sucrose) and reconstituted prior to mixing with the scaffold.

### Peptides/Peptide Complex

In some embodiments, the peptide complex includes two or more peptides selected to modulate one or more processes relevant to wound healing and scar prevention, including collagen synthesis and organization, MMP activity, inflammatory cytokine signaling, angiogenesis, mechanotransduction, and/or pigmentation.

In some embodiments, the peptide complex includes (i) one or more copper-binding/ECM-modulating peptides; (ii) one or more fibrosis-modulating/anti-TGF-β peptides; (iii) one or more mechanotransduction/contraction inhibitors; and/or (iv) one or more angiogenesis-modulating peptides. In some embodiments, the peptide composition further includes one or more pigmentation-regulating and depigmenting peptides. In some embodiments, the peptide composition further includes one or more wound healing peptides.

In some embodiments, the one or more copper-binding/ECM-modulating peptides are selected from GHK-Cu (copper tripeptide-1), lipidated GHK variants (Palmitoyl-GHK, Palmitoyl-GHK-Cu, Stearoyl-GHK) or matrixyl (palmitoyl pentapeptide-4).

In some embodiments, the one or more one or more fibrosis-modulating/anti-TGF-β peptides are selected from tetrapeptide-7, decorin-derived peptides (e.g., short peptides mimicking decorin's inhibitory effects on TGF-β); other TGF-β pathway modulators (e.g., peptides derived from connective tissue growth factor (CTGF) or small sequence motifs (identified via alanine scanning) that antagonize TGF-β receptor binding) with examples comprising LSKL (Leu-Ser-Lys-Leu) or P144 (Disitertide®).

In some embodiments, the one or more mechanotransduction or contraction inhibitors (e.g., selected from acetyl hexapeptide-8 (argireline); myristoyl hexapeptide-16, and related neuromodulatory peptides (SNAP-8, Acetyl Hexapeptide-8, Dipeptide Diaminobutyroyl Benzylamide Diacetate, Pentapeptide-18, Pentapeptide-3 and similar short peptides used in neuromodulation (anti-wrinkle applications) could share mechanistic overlap for reducing contractile forces in healing tissue).

In some embodiments, the one or more angiogenesis-modulating peptides are selected from oligopeptide-20 (VEGF-mimetic); QK peptide; and angiopoietin-derived peptides (e.g., short sequences extracted from angiopoietin proteins). Examples including Ac-QHREDGS, QHREDGS-NH₂, Ac-QHREDGS-NH₂, H-QHREDGS-NH₂, Ac-QHREDGS-OH (where QHREDGS has SEQ ID NO 8).

In some embodiments, the pigmentation-regulating and depigmenting peptides include oligopeptide-34 and tranexamic acid peptide conjugates and alpha-msh analogues and hexapeptide-9 (His-D-Phe-Arg-Trp-D-Phe-Lys) (melanostatine) (SEQ ID NO 9). Additional examples include Ac-IWSLDTQYGGRGD (SEQ ID NO 10), IWSLDTQYGGRGD-NH (SEQ ID NO 11), Ac-IWSLDTQYGGRGD-NH (SEQ ID NO 12), MPfRwFKPV-NH₂ (SEQ ID NO 13), Ac-MPfRwFKPV-NH₂ (SEQ ID NO 14).

In some embodiments, the one or more wound healing peptides are selected from thymosin beta-4 and self-assembling and scaffold-integrated peptides such as RADA16-I (Ac-RADARADARADARADA-NH₂) (SEQ ID NO 15), KLD-12 (Ac-KLDLKLDLKLDL-NH₂) (SEQ ID NO 16) and Q11 (Ac-QQKFQFQFEQQ-NH₂) (SEQ ID NO 17).

In some embodiments, the peptide component comprises two or more of GHK-Cu, Tetrapeptide-7, Palmitoyl-GHK, Palmitoyl Tripeptide-1, Acetyl Hexapeptide-8, Oligopeptide-20, Myristoyl Hexapeptide-16, and/or Oligopeptide-34, optionally in stabilized or modified forms (e.g., lipidated, PEGylated, cyclized, or D-amino acid substituted variants). In some embodiments, the peptide component comprises three or more of GHK-Cu, Tetrapeptide-7, Palmitoyl-GHK, Palmitoyl Tripeptide-1, Acetyl Hexapeptide-8, Oligopeptide-20, Myristoyl Hexapeptide-16, and/or Oligopeptide-34, optionally in stabilized or modified forms (e.g., lipidated, PEGylated, cyclized, or D-amino acid substituted variants). In some embodiments, the peptide component comprises four or more of GHK-Cu, Tetrapeptide-7, Palmitoyl-GHK, Palmitoyl Tripeptide-1, Acetyl Hexapeptide-8, Oligopeptide-20, Myristoyl Hexapeptide-16, and/or Oligopeptide-34, optionally in stabilized or modified forms (e.g., lipidated, PEGylated, cyclized, or D-amino acid substituted variants). In some embodiments, the peptide component comprises five or more of GHK-Cu, Tetrapeptide-7, Palmitoyl-GHK, Palmitoyl Tripeptide-1, Acetyl Hexapeptide-8, Oligopeptide-20, Myristoyl Hexapeptide-16, and/or Oligopeptide-34, optionally in stabilized or modified forms (e.g., lipidated, PEGylated, cyclized, or D-amino acid substituted variants).

In some embodiments, the peptide complex includes three of GHK-Cu, Tetrapeptide-7, Palmitoyl Tripeptide-1, Acetyl Hexapeptide-8, and Oligopeptide-20. In some embodiments, the peptide complex includes four of GHK-Cu, Tetrapeptide-7, Palmitoyl Tripeptide-1, Acetyl Hexapeptide-8, and Oligopeptide-20. In some embodiments, the peptide complex includes each of GHK-Cu, Tetrapeptide-7, Palmitoyl Tripeptide-1, Acetyl Hexapeptide-8, and Oligopeptide-20. In some embodiments, the peptide complex comprises GHK-Cu, Tetrapeptide-7, Palmitoyl Tripeptide-1, Acetyl Hexapeptide-8, and Oligopeptide-20 and at least one more peptide. In some embodiments, the peptide complex comprises GHK-Cu, Tetrapeptide-7, Palmitoyl Tripeptide-1, Acetyl Hexapeptide-8, and Oligopeptide-20 and at least two additional peptides.

In some embodiments, the peptide component comprises a copper-binding peptide such as GHK-Cu (copper tripeptide-1) at about 0.10% to about 3.00% w/w of the total composition.

In some embodiments, the peptide component includes an anti-fibrotic peptide such as Tetrapeptide-7 at about 0.05% to about 2.00% w/w (e.g., 0.50% to 2.00% w/w).

In some embodiments, the peptide component includes one or more lipidated peptides selected from Palmitoyl-GHK, Palmitoyl Tripeptide-1, Palmitoyl Pentapeptide-4, and Myristoyl Hexapeptide-16. It is believed that lipidation can improve dermal penetration and/or retention. Example lipids may include Palmitic acid (C16:0; hexadecanoic acid), Myristic acid (C14:0; tetradecanoic acid), and Stearic acid (C18:0; octadecanoic acid),

In some embodiments, the peptide component includes a mechanotransduction inhibitor peptide such as Acetyl Hexapeptide-8 at about 0.05% to about 2.00% w/w (e.g., 0.50% to 2.00% w/w), optionally combined with Myristoyl Hexapeptide-16 at about 0.001% to about 0.01% w/w.

In some embodiments, the peptide component includes an angiogenesis-modulating peptide such as Oligopeptide-20 (VEGF-mimetic) at about 0.10% to about 1.00% w/w (e.g., 0.25% to 1.00% w/w). In some embodiments, the peptide component further includes one or more pigmentation-regulating agents, including Oligopeptide-34 and/or tranexamic acid for post-procedural hyperpigmentation reduction.

In some embodiments, the peptides are included in base form or as stabilized variants, including lipidated forms, PEGylated forms, cyclized forms, D-amino acid substituted forms, N-methylated forms, and/or conjugates with carriers (e.g., liposomes, polymer conjugates, enzyme-responsive linkers). Specific examples may comprise Palmitoyl-GHK/Palmitoyl Tripeptide-1, Palmitoyl Pentapeptide-4, Myristoyl Hexapeptide-16, PEG-GHK, PEG-QHREDGS, PEG-Thymosin beta-4, Cyclo-QHREDGS, Disulfide-cyclized GHK analogs, QHRE^{D}DGS, N-Me-Gly-GHK N-methylated QHREDGS variants, Liposome-encapsulated Palmitoyl-GHK, Liposome-encapsulated Thymosin beta-4, Hyaluronic acid-GHK conjugate, and Protease-cleavable QHREDGS-hydrogel conjugate. some embodiments, exemplary peptide ranges in a topical composition include: GHK-Cu at about 0.01% to about 3.00% w/w; Tetrapeptide-7 at about 0.01% to about 2.00% w/w; Palmitoyl Tripeptide-1 at about 0.05% to about 5.00% w/w; Acetyl Hexapeptide-8 at about 0.01% to about 2.00% w/w; Oligopeptide-20 at about 0.01% to about 2.00% w/w; and Oligopeptide-34 at about 0.01% to about 3.00% w/w.

In some embodiments, peptides are modified to enhance stability and/or bioavailability, including by PEGylation (e.g., attachment of polyethylene glycol chains of varying length), alternative lipidation (e.g., palmitoyl, myristoyl, stearoyl, lauroyl, oleoyl), cyclization (including head-to-tail cyclization), side-chain stapling, D-amino acid substitution at non-critical positions, and/or selective N-methylation of peptide bonds.

In some embodiments, peptides are configured for improved delivery by fusion with cell-penetrating peptides (CPPs) (e.g., TAT, penetratin), by inclusion of integrin-targeting motifs (e.g., RGD), and/or by conjugation to biodegradable polymers (e.g., PLGA, hyaluronic acid) via cleavable linkages to enable sustained release from a hydrogel and/or scaffold. In certain embodiments, enzyme-responsive linkers are employed to release active peptides in response to wound-associated enzymes (e.g., MMP-sensitive sequences).

In some embodiments, structural optimization includes hydrocarbon stapling to stabilize α-helical conformations (e.g., for VEGF-mimetics), creation of fusion and/or bifunctional peptides (e.g., combining anti-inflammatory and ECM-remodeling domains), alanine scanning and/or rational mutagenesis to improve affinity and/or selectivity, and/or development of peptidomimetic constructs that preserve the relevant three-dimensional pharmacophore while improving pharmacokinetics.

In some embodiments, GHK-based peptides are configured as self-assembling conjugates to form nanostructures providing a depot effect, and/or as dual-function conjugates bearing antioxidant and/or anti-inflammatory moieties.

In some embodiments, Tetrapeptide-7 variants include sequence extensions, N- and/or C-terminal flanking modifications, and/or cyclized variants configured to stabilize an active conformation and/or increase anti-fibrotic potency.

In some embodiments, alternative acyl groups (e.g., benzoyl, isobutyryl) are employed to tune permeation and/or receptor interaction, and/or peptide dimerization is employed to increase functional activity in mechanotransduction pathways.

In some embodiments, cyclization and/or dimerization is employed to mimic VEGF multimeric structure while reducing off-target angiogenesis, and/or non-natural amino acids are incorporated to reinforce secondary structure and resist proteolysis.

In some embodiments, conjugation to tranexamic acid analogs is optimized by linker chemistry to provide controlled release, and/or hybrid peptides fuse depigmenting sequences with targeting domains to increase localization in melanocyte-rich regions.

### Placental ECM Scaffold

In some embodiments, the scaffold includes placental-derived extracellular matrix components obtained from placental tissue by decellularization and extraction, followed by processing to provide a hydrogel-forming or film-forming material. In some embodiments, the placental ECM includes one or more collagens (e.g., Type I, III, and/or IV), fibronectin, laminin, glycosaminoglycans, and/or residual growth factors.

In some embodiments, the scaffold includes heat-treated, decellularized, or otherwise processed placental ECM further includes one placental growth factors such as VEGF, PDGF, EGF, and/or HGF. In some embodiments, the one or more placental growth factors are each present at about 5 ng/mL to about 50 ng/mL in the scaffold.

In some embodiments, the scaffold forms an aqueous hydrogel. In other embodiments, the scaffold is incorporated into a gel-cream, emulsion cream, or other dermatologically acceptable topical base. In some embodiments, the hydrogel architecture can provide spatial co-localization of exosomes and peptides and enable temporally regulated diffusion of the active agents. In some embodiments, the scaffold is configured as (i) an in situ-gelling hydrogel, (ii) a pre-formed hydrogel sheet, (iii) an occlusive film, (iv) a patch or dressing impregnated with the actives, (v) a microneedle array with a dissolvable ECM matrix, or (vi) a sprayable or foaming formulation that gels on contact with skin.

### Optional Additional Components

In some embodiments, the composition further includes one or more additional agents selected from antioxidants, humectants, buffers, antimicrobials, barrier lipids, and soothing botanicals. Non-limiting examples include carnosine (e.g., 0.50% to 2.00% w/w), hyaluronic acid (e.g., 0.05% to 1.50% w/w depending on formulation), chitosan (e.g., 0.10% to 0.50% w/w), ceramides, niacinamide, centella asiatica extract, bisabolol, vitamin E, zinc PCA, and tranexamic acid, panthenol, allantoin, madecassoside, and combinations thereof.

In some embodiments, the pH of the final composition is adjusted to about 4.5 to about 7.5 (e.g., about 5.0 to about 6.5) using dermatologically acceptable buffers (e.g., citrate, phosphate, TRIS, disodium pyrophosphate (Na₂H₂P₂O₇) and tetrasodium pyrophosphate (Na₄P₂O₇)), 3-(N-morpholino)propanesulfonic acid (MOPS), N-(1,1-Dimethyl-2-hydroxyethyl)-3-amino-2-hydroxypropanesulfonic acid (AMPSO), trimethylamine, sodium 2,4-pentanedionate.

### Formulations

In some embodiments, the compositions of the present disclosure can be formulated as a hydrogel, gel, gel-cream, emulsion, ointment, lotion, spray, foam, patch, or impregnated dressing. In some embodiments, the exosome component and/or peptide complex are mixed into a pre-formed placental ECM hydrogel immediately prior to use to preserve activity. In some embodiments, the composition is sterile-filtered or aseptically filled.

### Formulations and Kits

In some embodiments, the composition is provided as a single-phase formulation in which exosomes and peptides are suspended or dispersed within a placental ECM hydrogel. In other embodiments, the composition is provided as a multi-part kit including (i) an exosome-containing component and (ii) a scaffold and/or peptide component, with instructions to combine immediately prior to application to preserve exosome activity.

Non-limiting exemplary formulations are provided below. Concentrations are expressed as mass percent (w/w) unless otherwise indicated. A vehicle/base is used to balance the formulation to 100%.

TABLE 1
Scar and Wound Healing Formulations

| Component | | Example 1 | Example 2 | Example 3 |
|---|---|---|---|---|
| Type/Class | Component | (mass %) | (Mass %) | (Mass %) |
| Regenerative | Placental | 1 billion per 30 | 50 billion per 30 | 100 billion per 30 |
| Signaling Agent | MSC-derived | mL | mL | ml |
| | exosomes | | | |
| Copper-Binding | GHK-Cu | 0.10% | 2.50% | 3.00% |
| Peptide | | | | |
| Anti-Fibrotic Peptide | Tetrapeptide-7 | 0.50% | 1.50% | 2.00% |
| ECM Remodeling | Palmitoyl | 1.00% | 2.50% | 3.00% |
| Peptide | Tripeptide-1 | | | |
| Mechanotransduction | Acetyl | 0.50% | 1.50% | 2.00% |
| Inhibitor Peptide | Hexapeptide-8 | | | |
| Angiogenesis | Oligopeptide-20 | 0.25% | 0.75% | 1.00% |
| Modulator Peptide | (VEGF-mimetic) | | | |
| Antioxidant Peptide | Carnosine | 0.50% | 1.50% | 2.00% |
| Hydration/ECM | Hyaluronic Acid | 0.05% | 0.20% | 0.30% |
| Stabilizer | | | | |
| Antimicrobial/Skin | Chitosan | 0.10% | 0.30% | 0.50% |
| Adhesion Agent | | | | |
| pH Stabilizer/Buffer | Sodium | 0.50% | 0.50% | 0.50% |
| | Citrate/Citric | | | |
| | Acid Buffer | | | |
| Vehicle/Base | Aqueous | about 95.6%* | about 88.75%* | about 85.7%* |
| | Gel/Emulsion | | | |
| | Cream Base | | | |
| | (balance to 100%) | | | |

TABLE 2
Post-Aesthetic Procedure Recovery

| Component | | Example 4 | Example 5 | Example 6 |
|---|---|---|---|---|
| Type/Class | Component | (mass %) | (Mass %) | (Mass %) |
| Barrier Repair & | Ceramide | 0.10% | 2.00% | 3.00% |
| Hydration | Complex (3:1:1 | | | |
| | Ratio) | | | |
| Barrier Repair & | Niacinamide | 2.00% | 4.00% | 6.00% |
| Hydration | | | | |
| Barrier Repair & | Hyaluronic Acid | 0.50% | 1.00% | 1.50% |
| Hydration | | | | |
| Anti-Inflammatory | MSC Exosomes | 1 billion per 30 | 50 billion per 30 | 100 billion per 30 |
| & Soothing | | mL | mL | mL |
| Anti-Inflammatory | Centella Asiatica | 1.00% | 2.00% | 3.00% |
| & Soothing | Extract | | | |
| Anti-Inflammatory | Bisabolol | 0.25% | 0.50% | 0.75% |
| & Soothing | | | | |
| Antioxidant & | GHK-Cu | 1.00% | 1.50% | 2.00% |
| Collagen Support | | | | |
| Antioxidant & | Palmitoyl | 1.50% | 2.00% | 2.50% |
| Collagen Support | Tripeptide-1 | | | |
| Antioxidant & | Vitamin E | 0.50% | 1.00% | 1.50% |
| Collagen Support | | | | |
| Hyperpigmentation | Oligopeptide-34 | 1.00% | 2.00% | 3.00% |
| Prevention | | | | |
| Hyperpigmentation | Tranexamic Acid | 2.00% | 3.00% | 4.00% |
| Prevention | | | | |
| Antimicrobial | Zinc PCA | 0.25% | 0.50% | 0.75% |
| Protection | | | | |
| Vehicle/Base | Gel-cream Base | about 89.0%* | about 80.5%* | about 72.0%* |
| | with Emulsifiers | | | |
| | (pH 5.5-6.0) | | | |

### Methods of Use

The compositions of the present disclosure can be administered topically to a subject (e.g., a mammalian subject, such as a human) in need thereof. In some embodiments, the composition is applied to an acute wound, surgical incision, abrasion, burn, or post-procedural treatment site (e.g., after laser resurfacing, microneedling, dermabrasion, radiofrequency, ultrasound, or chemical peeling). In some embodiments, the compositions of the present disclosure can be applied one or more times per day for a treatment period that can range from a single application to several weeks, depending on wound severity and clinical context.

In some embodiments, the composition is applied once, twice, or three times per day for a treatment period ranging from a single application to about 2-8 weeks depending on wound severity and clinical context. In some embodiments, the treated area is covered with an occlusive dressing for at least 1 hour, at least 4 hours, or overnight to increase retention.

In some embodiments, treatment reduces or prevents fibrotic scarring by modulating one or more of TGF-β/Smad signaling, NF-κB signaling, YAP/TAZ-mediated mechanotransduction, MMP/TIMP balance, collagen I/III ratio, and/or α-SMA expression. In some embodiments, treatment promotes controlled angiogenesis, reduces prolonged erythema, and supports improved pigmentation outcomes following procedures.

### Prophetic Examples

### Example 1 (Hydrogel Formulation)

A placental ECM hydrogel is prepared by decellularization, enzymatic solubilization, neutralization, and thermal gelation. An exosome suspension (e.g., 1×10\hat{ }10 particles/mL) is combined with a peptide complex comprising GHK-Cu, Tetrapeptide-7, Palmitoyl Tripeptide-1, Acetyl Hexapeptide-8, and Oligopeptide-20. The mixture is gently blended into the ECM hydrogel at 4-25° C. and filled into a sterile dispenser.

### Example 2 (Patch/Dressing)

The composition of Example 1 is loaded into a hydrogel sheet or non-woven dressing and sealed in a sterile pouch. The patch is applied to a post-laser resurfacing site immediately after the procedure and replaced once daily for 7-14 days.

### Example 3 (In Vitro Assays)

Keratinocyte scratch-wound assays and fibroblast collagen gel contraction assays are performed comparing (i) vehicle control, (ii) exosomes alone, (iii) peptide complex alone, and (iv) exosomes+peptide complex in placental ECM hydrogel. Endpoints include scratch closure rate, collagen contraction percentage, and gene expression levels of COL1A1, COL3A1, ACTA2 (α-SMA), TGFB1, IL6, and MMP1.

### Example 4 (Controlled Release)

Exosome and peptide release from crosslinked and non-crosslinked ECM hydrogels is measured in Franz diffusion cells. Release profiles are tuned by altering ECM concentration, crosslink density, and inclusion of co-scaffolds (e.g., hyaluronic acid, chitosan).

### Example 5

A composition comprising: a regenerative signaling agent comprising placental mesenchymal stem cell-derived exosomes at a concentration of 1 billion to 100 billion particles per 30 mL, a bioactive peptide complex including (a) GHK-Cu at 0.10% to 3.00% (mass percent), (b) Tetrapeptide-7 at 0.50% to 2.00%, (c) Palmitoyl Tripeptide-1 at 1.00% to 3.00%, (d) Acetyl Hexapeptide-8 at 0.50% to 2.00%, and (e) Oligopeptide-20 at 0.25% to 1.00%, wherein each peptide is optionally formulated in base or liposomal form; additional components including one or more of (a) Carnosine at 0.50% to 2.00%, (b) Hyaluronic Acid at 0.05% to 0.30%, (c) Chitosan at 0.10% to 0.50%, and (d) Sodium Citrate/Citric Acid Buffer at 0.50%; and a biocompatible scaffold including placental-derived Type I, III, and IV collagen, fibronectin, and laminin, incorporated into an aqueous gel or emulsion cream base, balancing the formulation to 100%. In some embodiments, the regenerative signaling agent comprises placental MSC-derived exosomes enriched with miRNAs including miR-21, miR-29, and miR-146a, unencapsulated and formulated for direct topical or scaffold-integrated administration. In some embodiments, the peptide complex comprises copper-binding, ECM-modulating, anti-fibrotic, mechanotransduction-inhibiting, and angiogenesis-modulating peptides within the defined ranges. In some embodiments, the scaffold comprises heat-treated or decellularized placental ECM, optionally integrated with hydrogel composites and configured for controlled release.

All the U.S. patents, U.S. patent application publications, U.S. patent applications, foreign patents, foreign patent applications, and non-patent publications referred to in this specification and/or listed in the Application Data Sheet are incorporated herein by reference, in their entirety. Aspects of the embodiments can be modified, if necessary to employ concepts of the various patents, applications, and publications to provide yet further embodiments.

Although the present disclosure has been described with reference to a number of illustrative embodiments, it should be understood that numerous other modifications and embodiments can be devised by those skilled in the art that will fall within the spirit and scope of the principles of this disclosure. More particularly, reasonable variations and modifications are possible in the component parts and/or arrangements of the subject combination arrangement within the scope of the foregoing disclosure, the drawings, and the appended claims without departing from the spirit of the disclosure. In addition to variations and modifications in the component parts and/or arrangements, alternative uses will also be apparent to those skilled in the art.

### Additional Embodiments

ADDITIONAL EMBODIMENT [1] Akit for post-procedure dermatologic recovery, comprising: (a) a first component comprising placental MSC-derived exosomes; (b) a second component comprising a peptide complex and a scaffold matrix; and (c) instructions for combining the first and second components immediately prior to application.

ADDITIONAL EMBODIMENT [2]A method of treating a cutaneous wound or reducing the risk of fibrotic scarring in a subject, comprising: topically applying the composition of claim 1 to the wound site, wherein the composition modulates inflammatory signaling, promotes extracellular matrix remodeling, and reduces TGF-beta and YAP/TAZ-mediated mechanotransduction pathways.

ADDITIONAL EMBODIMENT [3]A composition comprising: (a) a population of extracellular vesicles comprising exosomes; (b) a peptide complex comprising two or more peptides that modulate at least one of inflammation, extracellular matrix remodeling, mechanotransduction, angiogenesis, or pigmentation; and (c) a biocompatible scaffold comprising placental-derived extracellular matrix (ECM) components; wherein the exosomes and the peptide complex are co-localized within the scaffold.

ADDITIONAL EMBODIMENT [4] The composition of additional embodiment 3, wherein the exosomes are derived from mesenchymal stromal/stem cells (MSCs).

ADDITIONAL EMBODIMENT [5] The composition of additional embodiment 4, wherein the MSCs comprise placental-derived MSCs, umbilical cord-derived MSCs, Wharton's jelly MSCs, adipose-derived MSCs, or bone marrow-derived MSCs.

ADDITIONAL EMBODIMENT [6] The composition of additional embodiment 3, wherein the exosomes are enriched with one or more microRNAs selected from mi-R21, miR-29, and miR-146a.

ADDITIONAL EMBODIMENT [7] The composition of additional embodiment 3, wherein the exosomes are present in an amount ranging from about 1×10\hat{ }8 to about 5×10\hat{ }11 particles per mL.

ADDITIONAL EMBODIMENT [8] The composition of additional embodiment 3, wherein the exosomes are present in an amount ranging from between about 1×10\hat{ }9 to about 1×10\hat{ }11 particles per mL.

ADDITIONAL EMBODIMENT [9] The composition of additional embodiment 3, wherein the peptide complex comprises three or more peptides.

ADDITIONAL EMBODIMENT [10] The composition of additional embodiment 3, wherein the peptide complex comprises four or more peptides.

ADDITIONAL EMBODIMENT [11] The composition of additional embodiment 3, wherein the peptide complex comprises five or more peptides.

ADDITIONAL EMBODIMENT [12] The composition of additional embodiment 3, wherein the peptide complex comprises at least one copper-binding peptide selected from the group consisting of GHK-Cu and Palmitoyl-composition GHK.

ADDITIONAL EMBODIMENT [13] The composition of additional embodiment 3, wherein the peptide complex comprises at least one anti-fibrotic peptide selected from the group consisting of Tetrapeptide-7 and a decorin-mimetic peptide.

ADDITIONAL EMBODIMENT [14] The composition of additional embodiment 3, wherein the peptide complex comprises at least one mechanotransduction or contraction inhibitor peptide selected from the group consisting of Acetyl Hexapeptide-8 and Myristoyl Hexapeptide-16.

ADDITIONAL EMBODIMENT [15] The composition of additional embodiment 3, wherein the peptide complex comprises at least one angiogenesis-modulating peptide selected from the group consisting of Oligopeptide-20 and QK peptide.

ADDITIONAL EMBODIMENT [16] The composition of additional embodiment 3, wherein the peptide complex further comprises a pigmentation-regulating peptide selected from the group consisting of Oligopeptide-34 and Hexapeptide-9.

ADDITIONAL EMBODIMENT [17] The composition of additional embodiment 3, wherein the peptide complex comprises three or more peptides selected from the group consisting of GHK-Cu, Tetrapeptide-7, Palmitoyl Tripeptide-1, Palmitoyl Pentapeptide-4, Acetyl Hexapeptide-8, Oligopeptide-20, Myristoyl Hexapeptide-16, Oligopeptide-34, QK peptide, and thymosin-04.

ADDITIONAL EMBODIMENT [18]19. The composition of additional embodiment 3, wherein the peptide complex comprises three or more of GHK-Cu at about 0.01% to about 3.00% w/w; Tetrapeptide-7 at about 0.01% to about 2.00% w/w; Palmitoyl Tripeptide-1 at about 0.05% to about 5.00% w/w; Acetyl Hexapeptide-8 at about 0.01% to about 2.00% w/w; and Oligopeptide-20 at about 0.01% to about 2.00% w/w.

ADDITIONAL EMBODIMENT [19] The composition of additional embodiment 3, wherein two or more peptides in the peptide complex are lipidated, PEGylated, cyclized, D-amino-acid substituted, N-methylated, or encapsulated in liposomes.

ADDITIONAL EMBODIMENT [20]21. The composition of additional embodiment 3, wherein the placental-derived ECM components comprise one or more collagens selected from the group consisting of Type I collagen, Type III collagen, and Type IV collagen; and wherein the placental-derived ECM components further comprise fibronectin and laminin.

ADDITIONAL EMBODIMENT [21] The composition of additional embodiment 3, wherein the scaffold comprises a placental ECM-derived hydrogel.

ADDITIONAL EMBODIMENT [22] The composition of additional embodiment 21, further comprising one or more placental growth factors selected from the group consisting of VEGF, PDGF, EGF, and HGF.

ADDITIONAL EMBODIMENT [23] The composition of additional embodiment 21, wherein the hydrogel is crosslinked using genipin, EDC/NHS chemistry, transglutaminase, photo-crosslinking, or ionic crosslinking.

ADDITIONAL EMBODIMENT [24] The composition of additional embodiment 3, wherein the scaffold provides controlled release of the exosomes and peptides for at least 4 hours.

ADDITIONAL EMBODIMENT [25] The composition of additional embodiment 3, wherein the scaffold provides controlled release for at least 24 hours.

ADDITIONAL EMBODIMENT [26] The composition of additional embodiment 3, wherein the scaffold provides controlled release for at least 72 hours.

ADDITIONAL EMBODIMENT [27] The composition of additional embodiment 3, wherein the composition is formulated as a hydrogel, gel-cream, emulsion, ointment, lotion, spray, foam, or serum.

ADDITIONAL EMBODIMENT [28] The composition of additional embodiment 3, wherein the composition is formulated as a patch, film, or dressing impregnated with the exosomes and peptide complex.

ADDITIONAL EMBODIMENT [29] The composition of additional embodiment 3, further comprising one or more additional agents selected from antioxidants, humectants, antimicrobials, barrier lipids, pigmentation regulators, anesthetics, and soothing botanicals.

ADDITIONAL EMBODIMENT [30] The composition of additional embodiment 3, wherein the one or more additional agents comprise one or more of carnosine, hyaluronic acid, chitosan, ceramides, niacinamide, centella asiatica extract, bisabolol, vitamin E, zinc PCA, tranexamic acid, panthenol, and allantoin.

ADDITIONAL EMBODIMENT [31] The composition of additional embodiment 3, wherein the composition has a pH of about 4.5 to about 7.5.

ADDITIONAL EMBODIMENT [32]A method of treating a cutaneous wound in a subject, comprising topically applying the composition of additional embodiment 3 to a wound site.

ADDITIONAL EMBODIMENT [33] The method of additional embodiment 32, wherein the wound comprises a surgical incision, abrasion, laceration, burn, or chronic ulcer.

ADDITIONAL EMBODIMENT [34] The method of additional embodiment 32, wherein the wound site comprises a post-procedure site selected from a laser resurfacing site, microneedling site, dermabrasion site, radiofrequency treatment site, ultrasound treatment site, and chemical peel site.

ADDITIONAL EMBODIMENT [35] The method of additional embodiment 32, wherein the composition is applied within 0 to 24 hours after the procedure and then applied once or twice daily for at least 3 days.

ADDITIONAL EMBODIMENT [36] The method of additional embodiment 32, wherein application reduces one or more of TGF-β signaling, NF-κB signaling, YAP/TAZ signaling, α-SMA expression, or collagen gel contraction relative to an untreated control.

ADDITIONAL EMBODIMENT [37] The method of additional embodiment 32, wherein application reduces incidence or severity of hypertrophic scarring or keloid formation.

ADDITIONAL EMBODIMENT [38] The method of additional embodiment 32, wherein application reduces post-inflammatory hyperpigmentation or prolonged erythema following the procedure.

1. A composition for topical application to promote wound healing and scar reduction, the composition comprising: (a) a population of exosomes derived from mesenchymal stem cells (MSCs); (b) a peptide complex comprising two or more peptides selected from GHK-Cu, Tetrapeptide-7, Palmitoyl Tripeptide-1, Acetyl Hexapeptide-8, Oligopeptide-20, Myristoyl Hexapeptide-16, and Oligopeptide-34; and (c) a biocompatible scaffold comprising placental-derived extracellular matrix (ECM) components selected from Type I, Type III, and Type IV collagens, fibronectin, and laminin; wherein the exosomes and the peptide complex are co-localized within the scaffold.

2. The composition of claim 1, wherein the MSCs are enriched with one or more microRNAs selected from the group consisting of miR-21, miR-29, and miR-146a.

3. The composition of claim 1, wherein the exosomes are present in an amount ranging from between about 1×10⁹ to about 1×10¹¹ particles per milliliter.

4. The composition of claim 1, wherein the peptide complex comprises three or more of the peptides.

5. The composition of claim 1, wherein the peptide complex comprises four or more of the peptides.

6. The composition of claim 1, wherein the peptide complex includes: GHK-Cu at about 0.10% to 3.00% w/w; Tetrapeptide-7 at about 0.50% to 2.00% w/w; Palmitoyl Tripeptide-1 at about 1.00% to 3.00% w/w; Acetyl Hexapeptide-8 at about 0.50% to 2.00% w/w; and Oligopeptide-20 at about 0.25% to 1.00% w/w.

7. The composition of claim 1, wherein the scaffold further comprises one or more placental growth factors.

8. The composition of claim 7, wherein the one or more placetal growth factors are selected from the group consisting of VEGF, PDGF, EGF, and HGF.

9. The composition of claim 8, wherein the one or more placental growth factors are each present at about 5 ng/mL to about 50 ng/mL in the scaffold or the composition.

10. The composition of claim 1, wherein the scaffold is in the form of a hydrogel, gel-cream, emulsion, or other dermatologically acceptable topical base.

11. The composition of claim 1, wherein the scaffold is heat-treated or decellularized placental ECM providing controlled release of exosomes and peptides.

12. The composition of claim 1, further comprising one or more additional agents selected from: one or more antioxidants, one or more humectants, one or more antimicrobials, one or more barrier lipids, one or more pigmentation regulators, and one or moresoothing botanicals.

13. The composition of claim 12, wherein the one or more antioxidants is carnosine.

14. The composition of claim 12, wherein the one or more humectants is hyaluronic acid.

15. The composition of claim 12, wherein the one or more antimicrobials is chitosan.

16. The composition of claim 12, wherein the one or more barrier lipids is a ceramide.

17. The composition of claim 12, wherein the one or more pigmentation regulators are tranexamic acid or oligopeptide-34.

18. The composition of claim 12, wherein the one or more soothing botanticals are Centella asiatica extract or bisabolol.
